(19) United States
(12) Patent Application Publication
(10) Pub. No.: US 2026/0285851 A1
(43) Pub. Date: Sep. 24, 2026

(54) A PROCESS FOR THE PREPARATION OF PYROXASULFONE

(71) Applicant: GHARDA CHEMICALS LIMITED, Dombivli (IN)

(72) Inventors: Suchet Saran Mathur, Dombivli (IN); Huseni Fakhruddin Bookwala, Dombivli (IN); Narendra Narayan Vishe, Dombivli (IN); Mahesh Ashok Mahajan, Dombivli (IN); Pankaj Dnyandeo Lande, Dombivli (IN); Amar Ashok Khude, Dombivli (IN); Mayur Arvind Mali, Dombivli (IN)

(73) Assignee: GHARDA CHEMICALS LIMITED, Dombivli, MH (IN)

(21) Appl. No.: 19/472,528

(22) PCT Filed: Jan. 29, 2024
(86) PCT No.: PCT/IB2024/050801

§ 371 Date: Oct. 3, 2025

(30) Foreign Application Priority Data
Apr. 3, 2023 (IN) 202321025159
May 1, 2023 (IN) 202321030970
Jun. 17, 2023 (IN) 202321041310
Jul. 4, 2023 (IN) 202321044814

(51) Int. Cl. C07D 413/12

(52) U.S. Cl. CPC C07D 413/12

(57) ABSTRACT

The present disclosure relates to a process for the preparation of pyroxasulfone. The process steps for the preparation of pyroxasulfone proceeds under mild reaction conditions. The reagents used in the present disclosure are safe, can be recovered, and recycled. The process is simple, efficient, environment friendly, and provides pyroxasulfone with comparatively high purity and high yield.

### FIELD

The present disclosure relates to a process for the preparation of pyroxasulfone.

### BACKGROUND

The background information hereinbelow relates to the present disclosure but is not necessarily prior art.

Pyroxasulfone (CAS No. 447399-55-5) is a selective pre-emergence grass and broad-leaved weed herbicide that belongs to the oxazole/pyrazole class of herbicides. Pyroxasulfone is chemically known as 3-[[5-(difluoromethoxy)-1-methyl-3-(trifluoromethyl) pyrazole-4-yl]methylsulfonyl]-5,5-dimethyl-4H-isoxazole compound of Formula (I) and represented as:

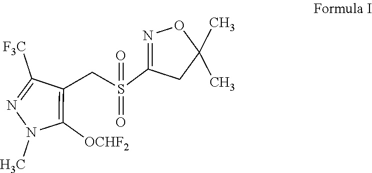 [SMILES: Cn1nc(C(F)(F)F)c(CS(=O)(=O)C2=NOC(C)(C)C2)c1OC(F)F]

Pyroxasulfone is used to control weeds such as foxtail, nutsedge, lamb's quarters, velvetleaf, ragweed, pigweed, and the like. Pyroxasulfone inhibits the very long-chain fatty acid elongase (VLCFAE) in the undesired weeds that grow along with crops such as corn, wheat, soybean, and the like. It provides high efficacy against broadleaf weeds even at low application rates.

Conventional methods for the preparation of Pyroxasulfone are associated with drawbacks such as impurities and a low yield of the product. These conventional processes thus require further purification of the crude product to remove impurities. The impurities in the product may affect the efficacy, safety, and stability of the final product and substantially increase the cost. Also, the yield and purity of Pyroxasulfone obtained from known processes is low.

Therefore, there is felt a need to provide a process for the preparation of Pyroxasulfone that mitigates the aforestated drawbacks or at least provides an alternative solution.

### Objects

Some of the objects of the present disclosure, which at least one embodiment herein satisfies, are as follows:

It is an object of the present disclosure to ameliorate one or more problems of the background or to at least provide a useful alternative.

Another object of the present disclosure is to provide a process for the preparation of Pyroxasulfone.

Yet another object of the present disclosure is to provide a process for the preparation of Pyroxasulfone with a comparatively better purity and yield.

Still another object of the present disclosure is to provide a simple and cost-effective process for the preparation of Pyroxasulfone.

Another object of the present disclosure is to provide an environment-friendly and commercially scalable process for the preparation of Pyroxasulfone.

Other objects and advantages of the present disclosure will be more apparent from the following description, which is not intended to limit the scope of the present disclosure.

### SUMMARY

The present disclosure relates to a process for the preparation of pyroxasulfone. The process comprises reacting ethyl-4,4,4-trifluoroacetoacetate (ETFAA) with a hydrazine salt in a first fluid medium by using a first base at a first predetermined temperature for a first predetermined time period to obtain 5-hydroxy-1-methyl-3-trifluoromethyl-1H-pyrazole. The 5-hydroxy-1-methyl-3-trifluoromethyl-1H-pyrazole is difluoromethylated by using a difluoromethylating agent and a second base in a second fluid medium at a second predetermined temperature for a second predetermined time period to obtain 5-(difluoromethoxy)-1-methyl-3-(trifluoromethyl)-1H-pyrazole. The 5-(difluoromethoxy)-1-methyl-3-(trifluoromethyl)-1H-pyrazole is chloromethylated by using a chloromethylating agent in the presence of a first catalyst at a third predetermined temperature for a third predetermined time period to obtain 4-(chloromethyl)-5-(difluoromethoxy)-1-methyl-3-(trifluoromethyl)-1H-pyrazole. Separately, glyoxylic acid is reacted with hydroxylamine and a halogenating agent at a fourth predetermined temperature for a fourth predetermined time period to obtain 1,1′-dibromoformaldoxime. The 1,1′-dibromoformaldoxime, a third base, and isobutylene in a third fluid medium are reacted at a fifth predetermined temperature for a fifth predetermined time period to obtain 3-bromo-5,5-dimethyl-4,5-dihydroisoxazole. The so obtained 3-bromo-5,5-dimethyl-4,5-dihydroisoxazole is reacted with thiourea in a fourth fluid medium in the presence of a second catalyst at a sixth predetermined temperature for a sixth predetermined time period to obtain 5,5-dimethyl-4,5-dihydroisoxazole-3-ylisothiourea hydrobromide. The 4-(chloromethyl)-5-(difluoromethoxy)-1-methyl-3-(trifluoromethyl)-1H-pyrazole is reacted with 5,5-dimethyl-4,5-dihydroisoxazole-3-ylisothiourea hydrobromide by using a fourth base and in a fifth fluid medium at a seventh predetermined temperature for a seventh predetermined time period to obtain 3-(((5-(difluoromethoxy)-1-methyl-3-(trifluoromethyl)-1H-pyrazol-4-yl)methyl)thio)-5,5-dimethyl-4,5-dihydroisoxazole compound. The 3-(((5-(difluoromethoxy)-1-methyl-3-(trifluoromethyl)-1H-pyrazol-4-yl)methyl)thio)-5,5-dimethyl-4,5-dihydroisoxazole is oxidized by using an oxidizing agent in the presence of a third catalyst in a sixth fluid medium at an eighth predetermined temperature for an eighth predetermined time period to obtain pyroxasulfone.

In an embodiment of the present disclosure, the hydrazine salt is at least one selected from the group consisting of methyl hydrazine sulphate and methyl hydrazine hydrochloride.

In an embodiment of the present disclosure, the first fluid medium is at least one selected from the group consisting of methanol, ethanol, propanol, and n-butanol.

In an embodiment of the present disclosure, the first predetermined temperature is in the range of 30° C. to 90° C.

In an embodiment of the present disclosure, the first predetermined time period is in the range of 4 hours to 8 hours.

In an embodiment of the present disclosure, the first base is at least one selected from the group consisting of triethylamine and diethylamine.

In an embodiment of the present disclosure, a mole ratio of the hydrazine salt to ethyl-4,4,4-trifluoroacetoacetate (ETFAA) is in the range of 1:1 to 1.5:1.

In an embodiment of the present disclosure, the second fluid medium is at least one selected from the group consisting of acetonitrile, methanol, ethanol, isopropyl alcohol, tetrahydrofuran (THF), dioxane, monoglyme, and diglyme.

In an embodiment of the present disclosure, the difluoromethylating agent is chlorodifluoromethane.

In an embodiment of the present disclosure, the second predetermined temperature is in the range of 20° C. to 45° C.

In an embodiment of the present disclosure, the second predetermined time period is in the range of 2 hours to 10 hours.

In an embodiment of the present disclosure, a mole ratio of the difluoromethylating agent to 5-hydroxy-1-methyl-3-trifluoromethyl-1H-pyrazole is in the range of 1:1 to 2.5:1.

In an embodiment of the present disclosure, the chloromethylating agent is a mixture of a chlorinating agent and an aldehyde.

In an embodiment of the present disclosure, the chlorinating agent is HCl.

In an embodiment of the present disclosure, the aldehyde is at least one selected from the group consisting of paraformaldehyde and formalin.

In an embodiment of the present disclosure, the first catalyst is at least one selected from the group consisting of methanesulphonic acid, monochloroacetic acid, dichloroacetic acid, trichloroacetic acid, benzenesulfonic acid, and p-toluene sulfonic acid.

In an embodiment of the present disclosure, the third predetermined temperature is in the range of 75° C. to 100° C.

In an embodiment of the present disclosure, the third predetermined time period is in the range of 10 hours to 20 hours.

In an embodiment of the present disclosure, a mole ratio of the chloromethylating agent to 5-(difluoromethoxy)-1-methyl-3-(trifluoromethyl)-1H-pyrazole is in the range of 6:1 to 7:1.

In an embodiment of the present disclosure, the halogenating agent is bromine.

In an embodiment of the present disclosure, the fourth predetermined temperature is in the range of −5° C. to 5° C.

In an embodiment of the present disclosure, the fourth predetermined time period is in the range of 4 hours to 6 hours.

In an embodiment of the present disclosure, the fifth predetermined temperature is in the range of 20° C. to 40° C.

In an embodiment of the present disclosure, the fifth predetermined time period is in the range of 1 hour to 2 hours.

In an embodiment of the present disclosure, the third base is at least one selected from the group consisting of sodium bicarbonate, potassium bicarbonate, potassium carbonate, and sodium carbonate.

In an embodiment of the present disclosure, the third fluid medium is at least one selected from the group consisting of water, methylene dichloride, and ethylene dichloride.

In an embodiment of the present disclosure, the fourth fluid medium is at least one selected from the group consisting of methanol, ethanol, isopropyl alcohol, and n-butanol.

In an embodiment of the present disclosure, the second catalyst is at least one selected from the group consisting of hydrogen bromide, and hydrogen chloride.

In an embodiment of the present disclosure, the sixth predetermined temperature is in the range of 20° C. to 40° C.

In an embodiment of the present disclosure, the sixth predetermined time period is in the range of 13 hours to 17 hours.

In an embodiment of the present disclosure, the fourth base is at least one selected from the group consisting of sodium hydroxide, potassium carbonate, and pyridine.

In an embodiment of the present disclosure, the fifth fluid medium is at least one selected from the group consisting of water and methanol.

In an embodiment of the present disclosure, the seventh predetermined temperature is in the range of 10° C. to 40° C.

In an embodiment of the present disclosure, the seventh predetermined time period is in the range of 3 hours to 35 hours.

In an embodiment of the present disclosure, a mole ratio of 5,5-dimethyl-4,5-dihydroisoxazole-3-ylisothiourea hydrobromide to 4-(chloromethyl)-5-(difluoromethoxy)-1-methyl-3-(trifluoromethyl)-1H-pyrazole is in the range of 1:1 to 1.5:1.

In an embodiment of the present disclosure, the third catalyst is at least one selected from the group consisting of phosphotungstic acid, sodium tungstate, methyl tri-n-octyl ammonium hydrogen sulphate, phenyl phosphonic acid, sodium phosphotungstate, phosphomolybdic acid, and silicotungstic acid.

In an embodiment of the present disclosure, the sixth fluid medium is at least one selected from the group consisting of dichloro acetic acid, monochloro acetic acid, acetic acid, pivalic acid, formic acid, aliphatic alcohol, and a mother liquor of chloroacetic acid.

In an embodiment of the present disclosure, the oxidizing agent is hydrogen peroxide.

In an embodiment of the present disclosure, the eighth predetermined temperature is in the range of 40° C. to 80° C.

In an embodiment of the present disclosure, the eighth predetermined time period is in the range of 2 hours to 20 hours.

In an embodiment of the present disclosure, a mole ratio of the oxidizing agent to 3-(((5-(difluoromethoxy)-1-methyl-3-(trifluoromethyl)-1H-pyrazol-4-yl)methyl)thio)-5,5-dimethyl-4,5-dihydroisoxazole is in the range of 2:1 to 5:1.

### DETAILED DESCRIPTION

The present disclosure relates to a process for the preparation of pyroxasulfone.

Embodiments, of the present disclosure, will now be described herein. Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise.

The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Conventional methods for the preparation of Pyroxasulfone are associated with drawbacks such as impurities and a low yield of the product. These conventional processes thus require further purification of the crude product to remove impurities. The impurities in the product may affect the efficacy, safety, and stability of the final product. Also, the yield and purity of Pyroxasulfone obtained from known processes is low.

The present disclosure provides an improved process for the preparation of Pyroxasulfone.

The process of the present disclosure is simple, environment friendly, economical, and results in improved yield and higher purity of Pyroxasulfone.

In an aspect of the present disclosure, there is provided a process for the preparation of Pyroxasulfone.

In an embodiment, a process for preparing Pyroxasulfone comprises the following steps:
a. reacting ethyl-4,4,4-trifluoroacetoacetate (ETFAA) with a hydrazine salt in a first fluid medium by using a first base at a first predetermined temperature for a first predetermined time period to obtain 5-hydroxy-1-methyl-3-trifluoromethyl-1H-pyrazole;
b. difluoromethylating the 5-hydroxy-1-methyl-3-trifluoromethyl-1H-pyrazole by using a difluoromethylating agent and a second base in a second fluid medium at a second predetermined temperature for a second predetermined time period to obtain 5-(difluoromethoxy)-1-methyl-3-(trifluoromethyl)-1H-pyrazole;
c. chloromethylating the 5-(difluoromethoxy)-1-methyl-3-(trifluoromethyl)-1H-pyrazole by using a chloromethylating agent in the presence of a first catalyst at a third predetermined temperature for a third predetermined time period to obtain 4-(chloromethyl)-5-(difluoromethoxy)-1-methyl-3-(trifluoromethyl)-1H-pyrazole;
d. separately reacting glyoxylic acid with hydroxylamine and a halogenating agent at a fourth predetermined temperature for a fourth predetermined time period to obtain 1,1′-dibromoformaldoxime;
e. reacting the 1,1′-dibromoformaldoxime, a third base, and isobutylene in a third fluid medium at a fifth predetermined temperature for a fifth predetermined time period to obtain 3-bromo-5,5-dimethyl-4,5-dihydroisoxazole;
f. reacting the 3-bromo-5,5-dimethyl-4,5-dihydroisoxazole with thiourea in a fourth fluid medium in the presence of a second catalyst at a sixth predetermined temperature for a sixth predetermined time period to obtain 5,5-dimethyl-4,5-dihydroisoxazole-3-ylisothiourea hydrobromide;
g. reacting the 4-(chloromethyl)-5-(difluoromethoxy)-1-methyl-3-(trifluoromethyl)-1H-pyrazole with the 5,5-dimethyl-4,5-dihydroisoxazole-3-ylisothiourea hydrobromide by using a fourth base in a fifth fluid medium at a seventh predetermined temperature for a seventh predetermined time period to obtain 3-(((5-(difluoromethoxy)-1-methyl-3-(trifluoromethyl)-1H-pyrazol-4-yl)methyl)thio)-5,5-dimethyl-4,5-dihydroisoxazole; and
h. oxidizing the 3-(((5-(difluoromethoxy)-1-methyl-3-(trifluoromethyl)-1H-pyrazol-4-yl)methyl)thio)-5,5-dimethyl-4,5-dihydroisoxazole by using an oxidizing agent in the presence of a third catalyst in a sixth fluid medium at an eighth predetermined temperature for an eighth predetermined time period to obtain pyroxasulfone.

The process is described in detail.

Firstly, a predetermined amount of a fluid medium is mixed with a hydrazine salt at a temperature in the range of 20° C. to 50° C. to obtain a first mixture. A predetermined amount of ethyl-4,4,4-trifluoroacetoacetate (ETFAA) is slowly added to the first mixture for a time period in the range of 60 minutes to 120 minutes to obtain a second mixture. To the second mixture, a predetermined amount of a first base is added for a time period in the range of 3 minutes to 120 minutes at a temperature in the range of 20° C. to 50° C. to obtain a reaction mass. The reaction mass is equilibrated at a first predetermined temperature for a first predetermined time period to obtain 5-hydroxy-1-methyl-3-trifluoromethyl-1H-pyrazole.

In an embodiment of the present disclosure, the first fluid medium is at least one selected from the group consisting of methanol, ethanol, propanol, and n-butanol. In an exemplary embodiment of the present disclosure, the first fluid medium is methanol.

In an embodiment of the present disclosure, the hydrazine salt is at least one selected from the group consisting of methyl hydrazine sulphate, and methyl hydrazine hydrochloride. In an exemplary embodiment of the present disclosure, the hydrazine salt is methyl hydrazine sulphate.

In an embodiment of the present disclosure, the first predetermined temperature is in the range of 30° C. to 90° C. In an exemplary embodiment of the present disclosure, the first predetermined temperature is 70° C.

In an embodiment of the present disclosure, the first predetermined time period is in the range of 4 hours to 8 hours. In an exemplary embodiment of the present disclosure, the first predetermined time period is 6 hours.

In an embodiment of the present disclosure, the first base is at least one selected from the group consisting of triethylamine, and diethylamine. In an exemplary embodiment of the present disclosure, the first base is triethylamine.

In an embodiment of the present disclosure, a mole ratio of the hydrazine salt to ethyl-4,4,4-trifluoroacetoacetate (ETFAA) is in the range of 1:1 to 1.5:1. In an exemplary embodiment of the present disclosure, the mole ratio of the hydrazine salt to ethyl-4,4,4-trifluoroacetoacetate (ETFAA) is 1.1:1.

In an embodiment of the present disclosure, the purity of 5-hydroxy-1-methyl-3-trifluoromethyl-1H-pyrazole is >99%.

In an embodiment of the present disclosure, the yield of 5-hydroxy-1-methyl-3-trifluoromethyl-1H-pyrazole is >80%.

In an embodiment of the present disclosure, the selectivity of 5-hydroxy-1-methyl-3-trifluoromethyl-1H-pyrazole is >99.8%.

A schematic representation for the preparation of 5-hydroxy-1-methyl-3-trifluoromethyl-1H-pyrazole is given as Scheme A:

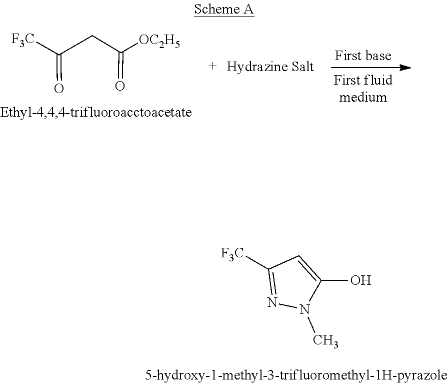

The present disclosure provides a simple and economical process for the preparation of 5-hydroxy-1-methyl-3-trifluoromethyl-1H-pyrazole with a comparatively higher yield, selectivity, and purity.

In an embodiment of the present disclosure, the first base and the first fluid medium used in the present disclosure can be easily recovered and recycled, hence the process is economical and environment friendly.

Further, a predetermined amount of 5-hydroxy-1-methyl-3-trifluoromethyl-1H-pyrazole is mixed with a second base under stirring at a temperature in the range of 20° C. to 45° C. for a time period in the range of 15 minutes to 60 minutes to obtain a first mixture. To the first mixture, a predetermined amount of a second fluid medium is added to obtain a second mixture. The second mixture is difluoromethylated by using a difluoromethylating agent at a second predetermined temperature for a second predetermined time period to obtain 5-(difluoromethoxy)-1-methyl-3-(trifluoromethyl)-1H-pyrazole.

In an embodiment of the present disclosure, the second base is at least one selected from the group consisting of sodium hydroxide, potassium hydroxide, sodium carbonate, and potassium carbonate. In an exemplary embodiment, the second base is sodium hydroxide. In another exemplary embodiment, the second base is potassium hydroxide. In still another exemplary embodiment, the second base is sodium carbonate. In yet another exemplary embodiment, the second base is potassium carbonate.

In an embodiment of the present disclosure, the second fluid medium is at least one selected from the group consisting of acetonitrile, methanol, ethanol, isopropyl alcohol, tetrahydrofuran (THF), dioxane, monoglyme, and diglyme. In an exemplary embodiment, the fluid medium is acetonitrile.

In an embodiment of the present disclosure, the difluoromethylating agent is chlorodifluoromethane.

In an embodiment of the present disclosure, the second predetermined temperature is in the range of 20° C. to 45° C. In an exemplary embodiment, the second predetermined temperature is 30° C.

In an embodiment of the present disclosure, the second predetermined time period is in the range of 2 hours to 10 hours. In an exemplary embodiment, the second predetermined time period is 5 hours.

In an embodiment of the present disclosure, a mole ratio of the difluoromethylating agent to 5-hydroxy-1-methyl-3-trifluoromethyl-1H-pyrazole is in the range of 1:1 to 2.5:1. In an exemplary embodiment of the present disclosure, the mole ratio of the difluoromethylating agent to 5-hydroxy-1-methyl-3-trifluoromethyl-1H-pyrazole is 2:1.

In an embodiment of the present disclosure, the purity of 5-(difluoromethoxy)-1-methyl-3-(trifluoromethyl)-1H-pyrazole is >95%.

In an embodiment of the present disclosure, the yield of 5-(difluoromethoxy)-1-methyl-3-(trifluoromethyl)-1H-pyrazole is >75%.

In an exemplary embodiment, a schematic representation for the preparation of 5-(difluoromethoxy)-1-methyl-3-(trifluoromethyl)-1H-pyrazole is provided hereinbelow as Scheme B.

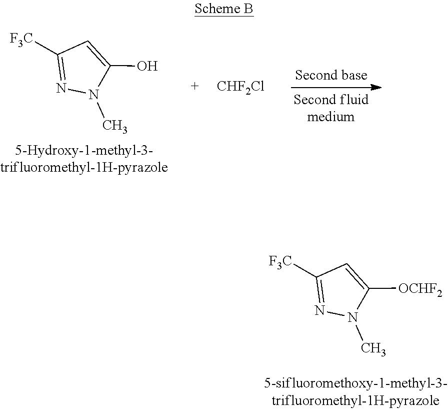

Still further, a predetermined amount of 5-(difluoromethoxy)-1-methyl-3-(trifluoromethyl)-1H-pyrazole is reacted with a chloromethylating agent in the presence of a first catalyst at a third predetermined temperature for a third predetermined time period to obtain 4-(chloromethyl)-5-(difluoromethoxy)-1-methyl-3-(trifluoromethyl)-1H-pyrazole.

In an embodiment of the present disclosure, the chloromethylating agent is a mixture of a chlorinating agent and an aldehyde.

In an embodiment of the present disclosure, the chlorinating agent is HCl.

In an embodiment of the present disclosure, the aldehyde is at least one selected from the group consisting of paraformaldehyde and formalin. In an exemplary embodiment, the aldehyde is paraformaldehyde.

In an embodiment of the present disclosure, the first catalyst is at least one selected from the group consisting of methanesulphonic acid, monochloroacetic acid dichloroacetic acid, trichloroacetic acid, benzenesulfonic acid and p-toluene sulfonic acid. In an exemplary embodiment, the first catalyst is methanesulphonic acid. In another exemplary embodiment, the first catalyst is monochloroacetic acid.

In an embodiment of the present disclosure, the third predetermined temperature is in the range of 75° C. to 100° C. In an exemplary embodiment, the third predetermined temperature is 85° C.

In an embodiment of the present disclosure, the third predetermined time period is in the range of 10 hours to 20 hours. In an exemplary embodiment, the third predetermined time period is 15 hours.

In an embodiment of the present disclosure, a mole ratio of the chloromethylating agent to 5-(difluoromethoxy)-1-methyl-3-(trifluoromethyl)-1H-pyrazole is in the range of 6:1 to 7:1. In an exemplary embodiment of the present disclosure, the mole ratio of the chloromethylating agent to 5-(difluoromethoxy)-1-methyl-3-(trifluoromethyl)-1H-pyrazole is 6.4:1.

In an embodiment of the present disclosure, the purity of 4-(chloromethyl)-5-(difluoromethoxy)-1-methyl-3-(trifluoromethyl)-1H-pyrazole is >90%.

In an embodiment of the present disclosure, the yield of 4-(chloromethyl)-5-(difluoromethoxy)-1-methyl-3-(trifluoromethyl)-1H-pyrazole is >80%.

In an exemplary embodiment, a schematic representation for the preparation of 4-(chloromethyl)-5-(difluoromethoxy)-1-methyl-3-(trifluoromethyl)-1H-pyrazole is provided hereinbelow as Scheme C.

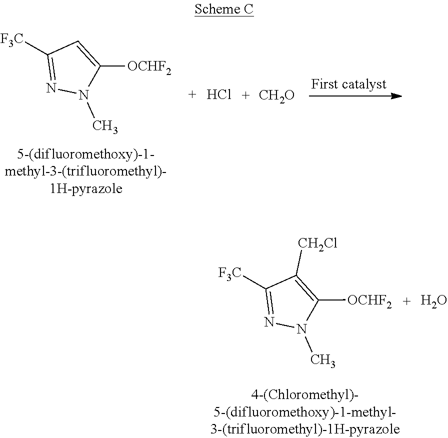

Separately glyoxylic acid is reacted with hydroxylamine and a halogenating agent at a fourth predetermined temperature for a fourth predetermined time period to obtain 1,1′-dibromoformaldoxime.

The process is described in detail.

A predetermined amount of glyoxylic acid solution is reacted with hydroxylamine sulphate at a temperature in the range of 20° C. to 40° C. for a time period in the range of 2 hours to 4 hours to obtain hydroxyiminoacetic acid. The hydroxyiminoacetic acid is reacted with a base to obtain a salt of hydroxyiminoacetic acid.

Maintaining the salt of hydroxyiminoacetic acid at a fourth predetermined temperature, a predetermined amount of a halogenating agent is slowly added, and the pH is maintained in the range of 3 to 4 by the addition of dilute sodium bicarbonate solution for a fourth predetermined time period to obtain 1,1′-dibromoformaldoxime.

In an embodiment of the present disclosure, the halogenating agent is bromine.

In an embodiment of the present disclosure, the fourth predetermined temperature is in the range of −5° C. to 5° C. In an exemplary embodiment of the present disclosure, the fourth predetermined temperature is 0° C.

In an embodiment of the present disclosure, the fourth predetermined time period is in the range of 4 hours to 6 hours. In an exemplary embodiment of the present disclosure, the fourth predetermined time period is 5 hours.

In an exemplary embodiment, a schematic representation for the preparation of 1,1′-dibromoformaldoxime is provided hereinbelow as Scheme D.

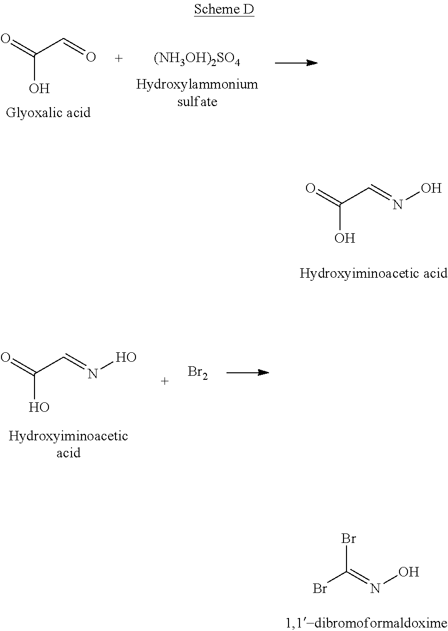

Further, predetermined amounts of a third base and a third fluid medium are mixed to obtain a first mixture. Maintaining the temperature in the range of 0° C. to 5° C., isobutylene gas is slowly passed for 1 hour to 2 hours to obtain a second mixture. Maintaining the temperature in the range of 0° C. to 5° C., 1,1′-dibromoformaldoxime (95% pure) solution prepared in methylene dichloride is slowly added to the second mixture to obtain a third mixture. To the third mixture, isobutylene gas is again purged at a temperature in the range of 0° C. to 5° C. to obtain a fourth mixture followed by heating at a fifth predetermined temperature for a fifth predetermined time period to obtain 3-bromo-5,5-dimethyl-4,5-dihydroisoxazole.

In an embodiment of the present disclosure, the fifth predetermined temperature is in the range of 20° C. to 40° C. In an exemplary embodiment of the present disclosure, the fifth predetermined temperature is 30° C.

In an embodiment of the present disclosure, the fifth predetermined time period is in the range of 1 hour to 2 hours. In an exemplary embodiment of the present disclosure, the fifth predetermined time period is 1.5 hours.

In an embodiment of the present disclosure, the third base is at least one selected from the group consisting of sodium bicarbonate, potassium bicarbonate, potassium carbonate, and sodium carbonate. In an exemplary embodiment of the present disclosure, the third base is sodium bicarbonate.

In an embodiment of the present disclosure, the third fluid medium is at least one selected from the group consisting of water, methylene dichloride, and ethylene dichloride. In an exemplary embodiment of the present disclosure, the third fluid medium is water.

In an exemplary embodiment, a schematic representation for the preparation of 3-bromo-5,5-dimethyl-4,5-dihydroisoxazole is provided hereinbelow as Scheme E.

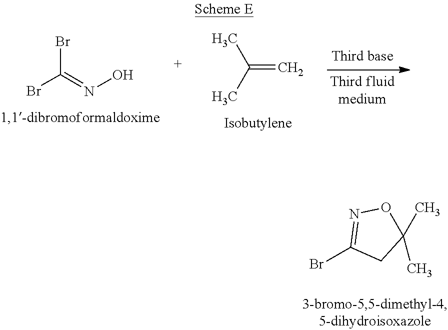

Still further, predetermined amounts of thiourea and a fourth fluid medium are mixed to obtain a first mixture. Maintaining the temperature in the range of 20° C. to 40° C., 3-bromo-5,5-dimethyl-4,5-dihydroisoxazole is added to the first mixture to obtain a second mixture. A predetermined amount of a second catalyst is added to the second mixture and heated at a sixth predetermined temperature for a sixth predetermined time period to obtain 5,5-dimethyl-4,5-dihydroisoxazole-3-ylisothiourea hydrobromide.

In an embodiment of the present disclosure, the fourth fluid medium is at least one selected from the group consisting of methanol, ethanol, isopropyl alcohol, and n-butanol. In an exemplary embodiment of the present disclosure, the fourth fluid medium is methanol.

In an embodiment of the present disclosure, the second catalyst is at least one selected from the group consisting of hydrogen bromide, and hydrogen chloride. In an exemplary embodiment of the present disclosure, the second catalyst is hydrogen bromide.

In an embodiment of the present disclosure, the sixth predetermined temperature is in the range of 20° C. to 40° C. In an exemplary embodiment of the present disclosure, the sixth predetermined temperature is 30° C.

In an embodiment of the present disclosure, the sixth predetermined time period is in the range of 13 hours to 17 hours. In an exemplary embodiment of the present disclosure, the sixth predetermined time period is 15 hours.

In an exemplary embodiment, a schematic representation for the preparation of 5,5-dimethyl-4,5-dihydroisoxazole-3-ylisothiourea hydrobromide is provided hereinbelow as Scheme F.

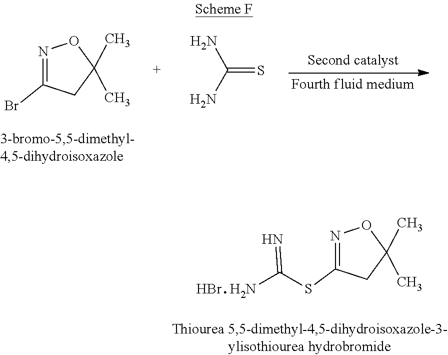

Furthermore, a predetermined amount of 4-(chloromethyl)-5-(difluoromethoxy)-1-methyl-3-(trifluoromethyl)-1H-pyrazole is reacted with 5,5-dimethyl-4,5-dihydroisoxazole-3-ylisothiourea hydrobromide by using a fourth base in a fifth fluid medium at a seventh predetermined temperature for a seventh predetermined time period to obtain 3-(((5-(difluoromethoxy)-1-methyl-3-(trifluoromethyl)-1H-pyrazol-4-yl)methyl)thio)-5,5-dimethyl-4,5-dihydroisoxazole.

In an embodiment of the present disclosure, the fourth base is at least one selected from the group consisting of sodium hydroxide, potassium carbonate, and pyridine. In an exemplary embodiment, the base is a mixture of sodium hydroxide and potassium carbonate with a predetermined weight ratio. In another embodiment, the base is a mixture of sodium hydroxide and pyridine with a predetermined weight ratio.

In an embodiment of the present disclosure, the fifth fluid medium is at least one selected from the group consisting of water and methanol. In an exemplary embodiment of the present disclosure, the fifth fluid medium is water. In another exemplary embodiment of the present disclosure, the fifth fluid medium is methanol.

In an embodiment of the present disclosure, the seventh predetermined temperature is in the range of 10° C. to 40° C. In an exemplary embodiment of the present disclosure, the seventh predetermined temperature is 25° C.

In an embodiment of the present disclosure, the seventh predetermined time period is in the range of 3 hours to 35 hours. In an exemplary embodiment of the present disclosure, the seventh predetermined time period is 30 hours. In another exemplary embodiment of the present disclosure, the seventh predetermined time period is 7 hours. In still another exemplary embodiment of the present disclosure, the seventh predetermined time period is 20 hours. In yet another exemplary embodiment of the present disclosure, the seventh predetermined time period is 4 hours.

In an embodiment of the present disclosure, a mole ratio of 5,5-dimethyl-4,5-dihydroisoxazole-3-ylisothiourea hydrobromide to 4-(chloromethyl)-5-(difluoromethoxy)-1-methyl-3-(trifluoromethyl)-1H-pyrazole is in the range of 1:1 to 1.5:1. In an exemplary embodiment of the present disclosure, the mole ratio of 5,5-dimethyl-4,5-dihydroisoxazole-3-ylisothiourea hydrobromide to 4-(chloromethyl)-5-(difluoromethoxy)-1-methyl-3-(trifluoromethyl)-1H-pyrazole is 1.1:1.

In an embodiment of the present disclosure, 3-(((5-(difluoromethoxy)-1-methyl-3-(trifluoromethyl)-1H-pyrazol-4-yl)methyl)thio)-5,5-dimethyl-4,5-dihydroisoxazole has a purity of >90%.

A schematic representation for the preparation of 3-(((5-(difluoromethoxy)-1-methyl-3-(trifluoromethyl)-1H-pyrazol-4-yl)methyl)thio)-5,5-dimethyl-4,5-dihydroisoxazole compounds is given as an exemplary embodiment in Scheme G.

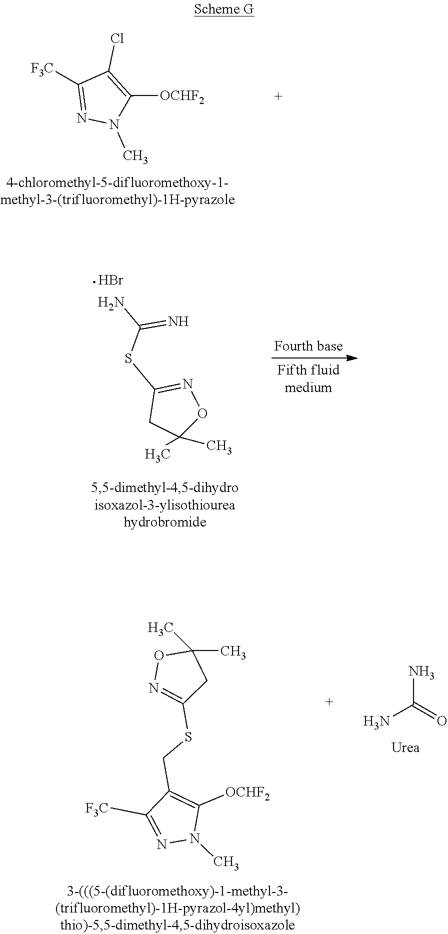

In a final step, a predetermined amount of 3-(((5-(difluoromethoxy)-1-methyl-3-(trifluoromethyl)-1H-pyrazol-4-yl)methyl)thio)-5,5-dimethyl-4,5-dihydroisoxazole is mixed with a sixth fluid medium in the presence of a third catalyst at a temperature in the range of 10° C. to 40° C. to obtain a reaction mixture. The reaction mixture is heated at a temperature in the range of 40° C. to 80° C. to obtain a heated reaction mixture. A predetermined amount of an oxidizing agent is added slowly to the heated reaction mixture for a time period in the range of 30 minutes to 300 minutes to obtain a reaction mass. The reaction mass is heated at an eighth predetermined temperature for an eighth predetermined time period to obtain a product mass of pyroxasulfone.

In an embodiment of the present disclosure, the third catalyst is at least one selected from the group consisting of phosphotungstic acid, sodium tungstate, methyl tri-n-octyl ammonium hydrogen sulphate, phenyl phosphonic acid, sodium phosphotungstate, phosphomolybdic acid, and silicotungstic acid. In an exemplary embodiment of the present disclosure, the third catalyst is phosphotungstic acid. In another exemplary embodiment of the present disclosure, the third catalyst is sodium tungstate. In still another exemplary embodiment of the present disclosure, the third catalyst is phenyl phosphonic acid.

In an embodiment of the present disclosure, the sixth fluid medium is at least one selected from the group consisting of dichloro acetic acid, monochloro acetic acid, acetic acid, pivalic acid, formic acid, aliphatic alcohol and a mother liquor of chloroacetic acid. In an exemplary embodiment of the present disclosure, the sixth fluid medium is the mother liquor of chloroacetic acid.

In an embodiment of the present disclosure, the aliphatic alcohol is at least one selected from the group consisting of methanol, ethanol, isopropanol, butanol, and pentanol.

In accordance with the embodiment of the present disclosure, the mother liquor/spent liquor of chloroacetic acid comprises:
35 mass % to 85 mass % of dichloro acetic acid;
35 mass % to 60 mass % of monochloro acetic acid;
1 mass % to 10 mass % of acetic acid; and
1 mass % to 10 mass % of water;
   wherein the mass % of each component is with respect to the total mass of the mother liquor/spent liquor of chloroacetic acid.

The use of the mother liquor/spent liquor of Chloroacetic acid containing mixtures of acetic acid, monochloroacetic acid, and dichloroacetic acid, as a fluid medium makes the process of the present disclosure a green process.

Further, the mother liquor of chloroacetic acid is an effluent generated during the production of chloroacetic acid and hence, is available cheaply in the market. The mother liquor of chloroacetic acid is an environmental waste and requires high processing costs for recyclability/disposal. Thus, the use of the mother liquor of chloroacetic acid (an environment waste/effluent) makes the process of the present disclosure cost-effective and environment friendly. Moreover, the effluent load is also minimized as the waste mother liquor of chloroacetic acid is utilized instead of dumping it into the environment.

In an embodiment of the present disclosure, the oxidizing agent is hydrogen peroxide.

In an embodiment of the present disclosure, the concentration of the oxidizing agent is in the range of 20% to 50%. In an exemplary embodiment of the present disclosure, the concentration of the oxidizing agent is 30%. In another exemplary embodiment of the present disclosure, the concentration of the oxidizing agent is 50%.

In an embodiment of the present disclosure, the eighth predetermined temperature is in the range of 40° C. to 80° C. In an exemplary embodiment of the present disclosure, the eighth predetermined temperature is 50° C. In another exemplary embodiment of the present disclosure, the eighth predetermined temperature is 67° C.

In an embodiment of the present disclosure, the eighth predetermined time period is in the range of 2 hours to 20 hours. In an exemplary embodiment of the present disclosure, the eighth predetermined time period is 5 hours. In another exemplary embodiment of the present disclosure, the eighth predetermined time period is 7.5 hours. In still another exemplary embodiment of the present disclosure, the eighth predetermined time period is 8 hours. In yet another exemplary embodiment of the present disclosure, the eighth predetermined time period is 13 hours. In an exemplary embodiment of the present disclosure, the eighth predetermined time period is 15 hours.

In an embodiment of the present disclosure, the mole ratio of the oxidizing agent to 3-(((5-(difluoromethoxy)-1-methyl-3-(trifluoromethyl)-1H-pyrazol-4-yl)methyl)thio)-5,5-dimethyl-4,5-dihydroisoxazole is in the range of 2:1 to 5:1. In an exemplary embodiment of the present disclosure, the mole ratio of the oxidizing agent to 3-(((5-(difluoromethoxy)-1-methyl-3-(trifluoromethyl)-1H-pyrazol-4-yl)methyl)thio)-5,5-dimethyl-4,5-dihydroisoxazole is 2.2:1. In another exemplary embodiment of the present disclosure, the mole ratio of the oxidizing agent to 3-(((5-(difluoromethoxy)-1-methyl-3-(trifluoromethyl)-1H-pyrazol-4-yl)methyl) thio)-5,5-dimethyl-4,5-dihydroisoxazole is 2.4:1. In yet another exemplary embodiment of the present disclosure, the mole ratio of the oxidizing agent to 3-(((5-(difluoromethoxy)-1-methyl-3-(trifluoromethyl)-1H-pyrazol-4-yl)methyl) thio)-5,5-dimethyl-4,5-dihydroisoxazole is 2.5:1. In still another exemplary embodiment of the present disclosure, the mole ratio of the oxidizing agent to 3-(((5-(difluoromethoxy)-1-methyl-3-(trifluoromethyl)-1H-pyrazol-4-yl)methyl) thio)-5,5-dimethyl-4,5-dihydroisoxazole is 3.7:1.

The product mass of pyroxasulfone is cooled followed by filtering and drying to obtain Pyroxasulfone.

In an embodiment of the present disclosure, the product mass is cooled to a temperature in the range of 10° C. to 40° C. to obtain a cooled product mass. The cooled product mass is filtered to obtain a cake. The cake is mixed in water followed by adding an aqueous solution of an inorganic salt to decompose an excess of oxidizing agent, if present, in the product mass.

The inorganic salt is selected from the group consisting of sodium thiosulphate, sodium sulphite, and sodium metabisulfite. In an exemplary embodiment of the present disclosure, the inorganic salt is sodium thiosulphate.

In accordance with the process of the present disclosure, the pyroxasulfone so obtained has a yield in the range of 80% to 95% and a purity in the range of 99% to 99.5%.

A schematic representation for the preparation of Pyroxasulfone is given as Scheme H:

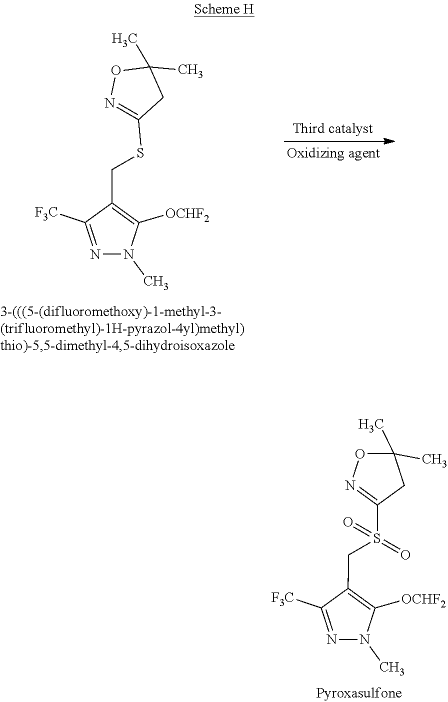 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

The foregoing description of the embodiments has been provided for purposes of illustration and is not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purposes only and not to be construed as limiting the scope of the disclosure. The following experiments are scalable to industrial/commercial process.

### EXPERIMENTAL DETAILS

### Experiment 1: Preparation of 5-hydroxy-1-methyl-3-trifluoromethyl-1H-pyrazole in Accordance with the Present Disclosure (Step a)

250 ml of methanol was charged into a reactor followed by adding 158.5 gm (1.1 mole) of methyl hydrazine sulphate at 30° C. to obtain a first mixture. Maintaining the temperature at 30° C., 192.0 gm (1.0 mole) of ethyl-4,4,4-trifluoroacetoacetate (ETFAA) was slowly added to the first mixture under stirring for 90 minutes to obtain a second mixture. To the second mixture, 113.4 gm (1.1 mole) of triethylamine (TEA) was slowly added under stirring for 60 minutes at 30° C. to obtain a reaction mass. The reaction mass was equilibrated at 30° C. for 30 minutes and heated to 70° C. for 6 hours to obtain a product mixture of 5-hydroxy-1-methyl-3-trifluoromethyl-1H-pyrazole with a conversion of >99%.

The product mixture was cooled to 50° C., followed by adding 500 ml of water to obtain a slurry. Methanol in the slurry was removed by distillation under a vacuum below 60° C. to obtain a white slurry. The so obtained white slurry was cooled to 10° C. and filtered to obtain a wet cake and a filtrate. The wet cake was washed with 500 ml of water and dried at 100° C. under vacuum to obtain 5-hydroxy-1-methyl-3-trifluoromethyl-1H-pyrazole.

The yield of 5-hydroxy-1-methyl-3-trifluoromethyl-1H-pyrazole was 84.6%, the purity was 99% and the selectivity was 99.8%.

Recovery of TEA from TEA-Sulphate in the Filtrate:

220 ml of ION NaOH was added to the filtrate for the pH adjustment up to 12 and converting the TEA sulphate in the filtrate to obtain a free base in a mixture. The mixture comprising free base was distilled at 80° C. to 95° C. to obtain a wet TEA (wet TEA contains some amount of water). The so obtained wet TEA was purified by fractionation to obtain TEA with a purity of >99.0% and moisture content of <0.1%. The overall recovery of TEA was >85%.

### Experiment 2A: Preparation of 5-(difluoromethoxy)-1-methyl-3-(trifluoromethyl)-1H-Pyrazole in Accordance with the Present Disclosure (Step b)

166.0 g (1.0 mole) of 5-hydroxy-1-methyl-3-trifluoromethyl-1H-pyrazole was charged into a reactor comprising 320 g (2.0 moles) of aqueous sodium hydroxide solution (25%) under stirring at 30° C. for 30 minutes to obtain a first mixture. 1200 ml of acetonitrile was mixed with the first mixture to obtain a second mixture. Maintaining the temperature at 30° C., 173 g (2.0 moles) of chlorodifluoromethane gas was passed into the second mixture in the reactor slowly for 3 hours, followed by stirring for 5 hours to obtain a biphasic mixture comprising an organic layer and an aqueous layer.

The organic layer was separated and concentrated under reduced pressure to obtain 5-(difluoromethoxy)-1-methyl-3-(trifluoromethyl)-1H-pyrazole.

The yield of 5-(difluoromethoxy)-1-methyl-3-(trifluoromethyl)-1H-pyrazole was 80%, and the purity was 97%.

### Experiment 2B: Preparation of 5-(difluoromethoxy)-1-methyl-3-(trifluoromethyl)-1H-pyrazole in Accordance with the Present Disclosure (Step b)

166.0 g (1.0 mole) of 5-hydroxy-1-methyl-3-trifluoromethyl-1H-pyrazole was charged into a reactor comprising 448 g (2.0 moles) of aqueous potassium hydroxide solution (25%) under stirring at 30° C. for 30 minutes to obtain a first mixture. 1200 ml of acetonitrile was mixed with the first mixture to obtain a second mixture. Maintaining the temperature at 30° C., 173 g (2.0 moles) of chlorodifluoromethane gas was passed into the second mixture in the reactor slowly for 3 hours, followed by stirring for 5 hours to obtain a biphasic mixture comprising an organic layer and an aqueous layer.

The organic layer was separated and concentrated under reduced pressure to obtain 5-(difluoromethoxy)-1-methyl-3-(trifluoromethyl)-1H-pyrazole.

The yield of 5-(difluoromethoxy)-1-methyl-3-(trifluoromethyl)-1H-pyrazole was 80%, and the purity was 98%.

### Experiment 2C: Preparation of 5-(difluoromethoxy)-1-methyl-3-(trifluoromethyl)-1H-pyrazole in Accordance with the Present Disclosure (Step b)

166.0 g (1.0 mole) of 5-hydroxy-1-methyl-3-trifluoromethyl-1H-pyrazole was charged into a reactor comprising 848 g (2.0 moles) of aqueous sodium carbonate solution (25%) under stirring at 30° C. for 30 minutes to obtain a first mixture. 1200 ml of acetonitrile was mixed with the first mixture to obtain a second mixture. Maintaining the temperature at 30° C., 173 g (2.0 moles) of chlorodifluoromethane gas was passed into the second mixture in the reactor slowly for 3 hours, followed by stirring for 5 hours to obtain a biphasic mixture comprising an organic layer and an aqueous layer.

The organic layer was separated and concentrated under reduced pressure to obtain 5-(difluoromethoxy)-1-methyl-3-(trifluoromethyl)-1H-pyrazole.

The yield of 5-(difluoromethoxy)-1-methyl-3-(trifluoromethyl)-1H-pyrazole was 75%, and the purity was 95%.

### Experiment 2D: Preparation of 5-(difluoromethoxy)-1-methyl-3-(trifluoromethyl)-1H-pyrazole in Accordance with the Present Disclosure (Step b)

166.0 g (1.0 mole) of 5-hydroxy-1-methyl-3-trifluoromethyl-1H-pyrazole was charged into a reactor comprising 1104 g (2.0 moles) of aqueous potassium carbonate solution (25%) under stirring at 30° C. for 30 minutes to obtain a first mixture. 1200 ml of acetonitrile was mixed with the first mixture to obtain a second mixture. Maintaining the temperature at 30° C., 173 g (2.0 moles) of chlorodifluoromethane gas was passed into the second mixture in the reactor slowly for 3 hours, followed by stirring for 5 hours to obtain a biphasic mixture comprising an organic layer and an aqueous layer.

The organic layer was separated and concentrated under reduced pressure to obtain 5-(difluoromethoxy)-1-methyl-3-(trifluoromethyl)-1H-pyrazole.

The yield of 5-(difluoromethoxy)-1-methyl-3-(trifluoromethyl)-1H-pyrazole was 75%, and the purity was 95%.

### Experiment 3A: Preparation of 4-(chloromethyl)-5-(difluoromethoxy)-1-methyl-3-(trifluoromethyl)-1H-pyrazole in Accordance with the Present Disclosure (Step c)

216 g (1.0 mole) of 5-difluoromethoxy-1-methyl-3-(trifluoromethyl)-1H-pyrazole was charged into a reactor followed by adding 608.3 g of aqueous HCl (30%) (5.0 moles), 42 g (1.4 moles) of paraformaldehyde and 19.2 g (0.2 mole) of methanesulphonic acid to obtain a reaction mixture. The reaction mixture was heated to 85° C. and maintained at 85° C. for 15 hours to obtain a resultant mixture. The reaction was monitored by HPLC for the presence of the starting material (<1%).

The resultant mixture was cooled to 30° C. to obtain a biphasic mixture comprising an organic layer and an aqueous layer. The organic layer was separated, washed with 200 ml of aqueous sodium chloride (10%), and dried over magnesium sulphate to obtain 4-(chloromethyl)-5-(difluoromethoxy)-1-methyl-3-(trifluoromethyl)-1H-pyrazole.

The yield of 4-(chloromethyl)-5-(difluoromethoxy)-1-methyl-3-(trifluoromethyl)-1H-pyrazole was 85% and the purity was 95%.

### Experiment 3B: Preparation of 4-(chloromethyl)-5-(difluoromethoxy)-1-methyl-3-(trifluoromethyl)-1H-pyrazole in Accordance with the Present Disclosure (Step c)

216 g (1.0 mole) of 5-difluoromethoxy-1-methyl-3-(trifluoromethyl)-1H-pyrazole was charged into a reactor followed by adding 608.3 g of aqueous HCl (30%) (5.0 moles), 42 g (1.4 moles) of paraformaldehyde and 50 ml of mother liquor of monochloroacetic acid to obtain a reaction mixture. The reaction mixture was heated to 85° C. and maintained at 85° C. for 18 hours to obtain a resultant mixture. The reaction was monitored by HPLC for the presence of the starting material (<1%).

The resultant mixture was cooled to 30° C. to obtain a biphasic mixture comprising an organic layer and an aqueous layer. The organic layer was separated, washed with 200 ml of aqueous sodium chloride (10%), and dried over magnesium sulphate to obtain 4-(chloromethyl)-5-(difluoromethoxy)-1-methyl-3-(trifluoromethyl)-1H-pyrazole.

The yield of 4-(chloromethyl)-5-(difluoromethoxy)-1-methyl-3-(trifluoromethyl)-1H-pyrazole was 82% and the purity was >92%.

### Experiment 4: Preparation of 1,1′-dibromoformaldoxime in Accordance with the Present Disclosure (Step d)

148 gm (1.0 mole) of 50.0% glyoxylic acid solution was charged into a reactor followed by slowly adding 82 gm of hydroxylamine sulphate (0.5 mole) in 175 g of water dropwise under stirring at 30° C. for 1 hour to obtain a first mixture. The first mixture was equilibrated at 30° C. for 90 minutes to obtain hydroxyiminoacetic acid. To the hydroxyiminoacetic acid, 187.5 ml of ION NaOH was added at 20° C. to obtain a sodium salt of hydroxyiminoacetic acid (pH=3.5). The sodium salt of hydroxyiminoacetic acid was cooled to 0° C. and equilibrated for 30 minutes.

Maintaining the sodium salt of hydroxyiminoacetic acid at 0° C., 320 gm (2.0 mole) of bromine was slowly added while simultaneously maintaining pH in the range of 3 to 4 by adding dilute sodium bicarbonate solution to obtain a reaction mass. The reaction mass was stirred for 5 hours at 0° C. to obtain a resultant mass.

The excess bromine in the resultant mass was decomposed by the addition of sodium thiosulphate solution followed by adding 500 ml of methylene dichloride and 200 ml of water to obtain a biphasic mixture comprising an organic phase and aqueous phase. The organic layer was separated and concentrated to obtain 1,1′-dibromoformaldoxime (white coloured solid).

The yield of 1,1′-dibromoformaldoxime was 75% and the purity was 96%.

### Experiment 5: Preparation of 3-bromo-5,5-dimethyl-4,5-dihydroisoxazole in Accordance With the Present Disclosure (Step e)

400 ml of water and 100.8 g (1.2 mole) of NaHCO₃ were charged into a reactor at 30° C. followed by adding 1000 ml of methylene dichloride to obtain a first mixture. The first mixture was cooled to 0° C. and maintaining the temperature in the range of 0° C. to 5° C., 112.0 g (2.0 mole) isobutylene gas slowly passed for 90 minutes to obtain a second mixture.

Maintaining the temperature in the range of 0° C. to 5° C., 213.7 gm (1.0 mole) of 1,1′-dibromoformaldoxime (95% pure) prepared in 1250 ml methylene dichloride solution was slowly added to the second mixture to obtain a third mixture. The third mixture was equilibrated at 0° C. for 1 hour followed by purging 56.0 gm (1.0 mole) of isobutylene gas at 0° C. to obtain a fourth mixture. The fourth mixture was equilibrated at 0° C. for 1 hour and then the temperature was raised to 30° C. over 90 minutes to obtain a biphasic mixture comprising an organic layer and an aqueous layer. The organic layer was concentrated completely to obtain 3-bromo-5,5-dimethyl-4,5-dihydroisoxazole.

The yield of 3-bromo-5,5-dimethyl-4,5-dihydroisoxazole was 76% and the purity was 96%.

### Experiment 6: Preparation of 5,5-dimethyl-4,5-dihydroisoxazole-3-ylisothiourea hydrobromide in Accordance with the Present Disclosure (Step f)

76.0 gm of (1.0 mole) thiourea and 500 ml of methanol were charged into a reactor at 30° C. to obtain a first mixture. Maintaining the temperature at 30° C., 187.4 gm (1.0 mole) of 95% pure 3-bromo-5,5-dimethyl-4,5-dihydroisoxazole was added to the first mixture to obtain a second mixture. To the second mixture, 33.75 g (0.2 mole) of aqueous hydrobromide solution was added and heated at 30° C. for 15 hours to obtain 5,5-dimethyl-4,5-dihydroisoxazole-3-ylisothiourea hydrobromide.

The yield of 5,5-dimethyl-4,5-dihydroisoxazole-3-ylisothiourea hydrobromide was 92%.

### Experiment 7A: Preparation of 3-(((5-(difluoromethoxy)-1-methyl-3-(trifluoromethyl)-1H-pyrazol-4-yl)methyl)thio)-5,5-dimethyl-4,5-dihydroisoxazole in Accordance with Present Disclosure (Step g)

12 g (0.3 mole) of NaOH flakes and 786.6 g (5.7 mole) of K₂CO₃ were added into 800 ml water followed by cooling to 20° C. to obtain an alkaline solution. Separately, 279.4 g (1.1 mole) of 5,5-dimethyl-4,5-dihydroisoxazole-3-ylisothiourea hydrobromide was dissolved in 760 ml water to obtain a solution. The solution of 5,5-dimethyl-4,5-dihydroisoxazole-3-ylisothiourea hydrobromide was added to the alkaline solution over a period of 1 hr to obtain a first mixture. Further, 264.5 g (1.0 mole) of 4-(chloromethyl)-5-(difluoromethoxy)-1-methyl-3-(trifluoromethyl)-1H-pyrazole was added to the first mixture over a period of 1 hr to obtain a second mixture. Then the temperature of the second mixture was raised to 25° C. and maintained at 25° C. for 30 hrs to obtain a reaction mixture comprising an organic phase and an aqueous phase. The reaction was monitored by HPLC. After 30 hrs, HPLC analysis showed <5% of 4-(chloromethyl)-5-(difluoromethoxy)-1-methyl-3-(trifluoromethyl)-1H-pyrazole. The organic phase was separated from the reaction mixture to obtain a separated organic phase and a separated aqueous phase. The separated organic phase was washed with 10% aqueous sodium chloride solution, further separated the organic phase, and was dried over magnesium sulphate to obtain a crude 3-[(5-difluoromethoxy-1-methyl-3-trifluoromethylpyrazol-4yl)-methyl thio)]-4,5-dihydro 5,5-dimethyl-isoxazole.

The yield of the crude of 3-(((5-(difluoromethoxy)-1-methyl-3-(trifluoromethyl)-1H-pyrazol-4-yl)methyl)thio)-5,5-dimethyl-4,5-dihydroisoxazole was 80% with 96% purity.

### Experiment 7B: Preparation of 3-(((5-(difluoromethoxy)-1-methyl-3-(trifluoromethyl)-1H-pyrazol-4-yl)methyl)thio)-5,5-dimethyl-4,5-dihydroisoxazole in Accordance with Present Disclosure (Step g)

12 g (0.3 mole) of NaOH flakes and 786.6 g (5.7 mole) of K₂CO₃ were added into 800 ml methanol followed by cooling to 20° C. to obtain an alkaline solution. Separately, 279.4 g (1.1 mole) of 5,5-dimethyl-4,5-dihydroisoxazole-3-ylisothiourea hydrobromide was dissolved in 760 ml methanol to obtain a solution. The solution of 5,5-dimethyl-4,5-dihydroisoxazole-3-ylisothiourea hydrobromide was added to the alkaline solution over a period of 1 hr to obtain a first mixture. Further, 264.5 g (1 mole) of 4-(chloromethyl)-5-(difluoromethoxy)-1-methyl-3-(trifluoromethyl)-1H-pyrazole was added to the first mixture over a period of 1 hr to obtain a second mixture. Then the temperature of the second mixture was raised to 25° C. and maintained at 25° C. for 7 hrs to obtain a reaction mixture. The reaction was monitored by HPLC. After 7 hrs, HPLC analysis showed <0.5% of 4-(chloromethyl)-5-(difluoromethoxy)-1-methyl-3-(trifluoromethyl)-1H-pyrazole.

1500 ml of methylene dichloride and 1500 ml of water were added to the reaction mixture to obtain a biphasic mixture comprising an organic phase and an aqueous phase. The organic phase and the aqueous phase were separated to obtain a separated organic phase and a separated aqueous phase. The separated aqueous layer was further extracted with 200 ml methylene dichloride and further separated a methylene dichloride layer. Both the separated methylene dichloride layer (organic phase) was dried over magnesium sulphate to obtain 3-(((5-(difluoromethoxy)-1-methyl-3-(trifluoromethyl)-1H-pyrazol-4-yl)methyl)thio)-5,5-dimethyl-4,5-dihydroisoxazole. Further, the separated organic phase was concentrated to remove the solvent (methylene dichloride) under reduced pressure to obtain a crude 3-(((5-(difluoromethoxy)-1-methyl-3-(trifluoromethyl)-1H-pyrazol-4-yl)methyl)thio)-5,5-dimethyl-4,5-dihydroisoxazole.

The yield of the crude of 3-(((5-(difluoromethoxy)-1-methyl-3-(trifluoromethyl)-1H-pyrazol-4-yl)methyl)thio)-5,5-dimethyl-4,5-dihydroisoxazole was 89% with 99% purity.

### Experiment 7C: Preparation of 3-(((5-(difluoromethoxy)-1-methyl-3-(trifluoromethyl)-1H-pyrazol-4-yl)methyl)thio)-5,5-dimethyl-4,5-dihydroisoxazole in Accordance with Present Disclosure (Step g)

152 g (3.8 mole) of NaOH flakes and 27.6 g (0.2 mole) of K₂CO₃ were added into 800 ml water followed by cooling to 20° C. to obtain an alkaline solution. Separately, 279.4 g (1.1 mole) of 5,5-dimethyl-4,5-dihydroisoxazole-3-ylisothiourea hydrobromide was dissolved in 760 ml water to obtain a solution. The solution of 5,5-dimethyl-4,5-dihydroisoxazole-3-ylisothiourea hydrobromide was added to the alkaline solution over a period of 1 hr to obtain a first mixture. Further, 264.5 g (1 mole) of 4-(chloromethyl)-5-(difluoromethoxy)-1-methyl-3-(trifluoromethyl)-1H-pyrazole was added to the first mixture over a period of 1 hr to obtain a second mixture. Then the temperature of the second mixture was raised to 25° C. and maintained at 25° C. for 20 hrs to obtain a reaction mixture comprising an organic phase and an aqueous phase. The reaction was monitored by HPLC. After 20 hrs, HPLC analysis showed <5% of 4-(chloromethyl)-5-(difluoromethoxy)-1-methyl-3-(trifluoromethyl)-1H-pyrazole. The organic phase and the aqueous phase were separated from the reaction mixture to obtain a separated organic phase and a separated aqueous phase. The separated organic phase was washed with 10% aqueous sodium chloride solution further separated the organic phase and was dried over magnesium sulphate to obtain a crude 3-[(5-difluoromethoxy-1-methyl-3-trifluoromethylpyrazol-4yl)-methyl thio)]-4,5-dihydro 5,5-dimethyl-isoxazole.

The yield of the crude of 3-(((5-(difluoromethoxy)-1-methyl-3-(trifluoromethyl)-1H-pyrazol-4-yl)methyl)thio)-5,5-dimethyl-4,5-dihydroisoxazole was 73% and purity was 93%.

### Experiment 7D: Preparation of 3-(((5-(difluoromethoxy)-1-methyl-3-(trifluoromethyl)-1H-pyrazol-4-yl)methyl)thio)-5,5-dimethyl-4,5-dihydroisoxazole in Accordance with Present Disclosure (Step g)

152 g (3.8 mole) of NaOH flakes and 27.6 g (0.2 mole) of K₂CO₃ were added into 800 ml methanol followed by cooling to 20° C. to obtain an alkaline solution. Separately, 279.4 g (1.1 mole) of 5,5-dimethyl-4,5-dihydroisoxazole-3-ylisothiourea hydrobromide was dissolved in 760 ml methanol to obtain a solution. The solution of 5,5-dimethyl-4,5-dihydroisoxazole-3-ylisothiourea hydrobromide was added to the alkaline solution over a period of 1 hr to obtain a first mixture. Further, 264.5 g (1.0 mole) of 44-(chloromethyl)-5-(difluoromethoxy)-1-methyl-3-(trifluoromethyl)-1H-pyrazole was added to the first mixture over a period of 1 hr to obtain a second mixture. Then the temperature of the second mixture was raised to 25° C. and maintained at 25° C. for 7 hrs to obtain a reaction mixture. The reaction was monitored by HPLC. After 7 hrs, HPLC analysis showed <0.5% of 4-(chloromethyl)-5-(difluoromethoxy)-1-methyl-3-(trifluoromethyl)-1H-pyrazole.

1500 ml methylene dichloride and 1500 ml water were added to the reaction mixture to obtain a biphasic mixture comprising an organic phase and an aqueous phase. The organic phase and the aqueous phase were separated to obtain a separated organic phase and a separated aqueous phase. The separated organic phase was dried over magnesium sulphate, and further separated the organic phase to obtain an organic phase containing a crude 3-(((5-(difluoromethoxy)-1-methyl-3-(trifluoromethyl)-1H-pyrazol-4-yl)methyl)thio)-5,5-dimethyl-4,5-dihydroisoxazole. The organic phase was concentrated to remove the solvent (methylene dichloride) under reduced pressure.

The yield of the crude 3-(((5-(difluoromethoxy)-1-methyl-3-(trifluoromethyl)-1H-pyrazol-4-yl)methyl)thio)-5,5-dimethyl-4,5-dihydroisoxazole was 95% and purity was 98%.

### Experiment 7E: Preparation of 3-(((5-(difluoromethoxy)-1-methyl-3-(trifluoromethyl)-1H-pyrazol-4-yl)methyl)thio)-5,5-dimethyl-4,5-dihydroisoxazole in Accordance with Present Disclosure (Step g)

152 g (3.8 mole) of NaOH flakes and 15.8 g (0.2 mole) of pyridine were added into 800 ml methanol followed by cooling to 20° C. to obtain an alkaline solution. Separately, 279.4 g (1.1 mole) of 5,5-dimethyl-4,5-dihydroisoxazole-3-ylisothiourea hydrobromide was dissolved in 760 ml methanol to obtain a solution. The solution of 5,5-dimethyl-4,5-dihydroisoxazole-3-ylisothiourea hydrobromide was added to the alkaline solution over a period of 1 hr to obtain a first mixture. Further, 264.5 g (1 mole) of 4-(chloromethyl)-5-(difluoromethoxy)-1-methyl-3-(trifluoromethyl)-1H-pyrazole was added to the first mixture over a period of 1 hr to obtain a second mixture. Then the temperature of the second mixture was raised to 25° C. and maintained at 25° C. for 4 hrs to obtain a reaction mixture. The reaction was monitored by HPLC. After 4 hrs, HPLC analysis showed <0.5% of 4-(chloromethyl)-5-(difluoromethoxy)-1-methyl-3-(trifluoromethyl)-1H-pyrazole.

1500 ml methylene dichloride and 1500 ml water were added to the reaction mixture to obtain a biphasic mixture comprising an organic phase and an aqueous phase. The organic phase and the aqueous phase were separated to obtain a separated organic phase (methylene dichloride) and a separated aqueous phase. The aqueous phase was further extracted with 200 ml methylene dichloride and further separated the methylene dichloride layer. Both methylene dichloride layers were combined and washed with 1N HCl solution, further washed the organic phase with water, and dried over magnesium sulphate to obtain a moisture free 3-[(5-difluoromethoxy-1-methyl-3-trifluoromethylpyrazol-4yl)-methyl thio)]-4,5-dihydro 5,5-dimethyl-isoxazole. The organic phase was concentrated to remove the methylene dichloride under reduced pressure to obtain a crude 3-(((5-(difluoromethoxy)-1-methyl-3-(trifluoromethyl)-1H-pyrazol-4-yl)methyl)thio)-5,5-dimethyl-4,5-dihydroisoxazole.

The yield of the crude 3-(((5-(difluoromethoxy)-1-methyl-3-(trifluoromethyl)-1H-pyrazol-4-yl)methyl)thio)-5,5-dimethyl-4,5-dihydroisoxazole was 89% and purity was 99%.

### Experiment 8A: Preparation of Pyroxasulfone from 3-(((5-(difluoromethoxy)-1-methyl-3-(trifluoromethyl)-1H-pyrazol-4-yl)methyl)thio)-5,5-dimethyl-4,5-dihydroisoxazole Using Phosphotungstic Acid as Catalyst and Methanol as a Fluid Medium (Step h)

A reactor was charged with 2.5 litre of methanol followed by adding 359 g (1 mole) of 3-(((5-(difluoromethoxy)-1-methyl-3-(trifluoromethyl)-1H-pyrazol-4-yl)methyl)thio)-5,5-dimethyl-4,5-dihydroisoxazole and 10 g of Phosphotungstic acid at 20° C. to obtain a reaction mixture. The reaction mixture was heated to 50° C. to obtain a heated reaction mixture. An aqueous solution of 30% hydrogen peroxide (249.3 g, 2.2 mole) was added dropwise to the heated reaction mixture over 1 hour to obtain a reaction mass. The reaction mass was heated to 50° C. under stirring and continued stirring at 50° C. for 15 hours to obtain a product mass. The pH of the product mass was adjusted to 8 by using 5% NaOH solution at 50° C. After completion of the reaction, an aqueous solution of sodium thiosulphate was added to the product mass to decompose excess hydrogen peroxide followed by adding 400 ml water slowly to obtain a resultant product mass. The resultant product mass was heated to 70° C. under stirring for 30 minutes followed by cooling to 10° C. and filtered to obtain a cake. The cake was washed with methanol followed by water to obtain a solid product. The solid product was dried under vacuum at 80° C. to obtain pyroxasulfone

The yield of pyroxasulfone was 90% and the purity was 99%.

### Experiment 8B: Preparation of Pyroxasulfone from 3-[[5-(difluoromethoxy)-1-methyl-3-(trifluoromethyl)pyrazole-4-yl]methylsulfanyl]-5,5-dimethyl-4H-isoxazole Using Sodium Tungstate as Catalyst and Methanol as a Fluid Medium (Step h)

A reactor was charged with 2.5 litre methanol followed by adding 359 g (1 mole) of 3-(((5-(difluoromethoxy)-1-methyl-3-(trifluoromethyl)-1H-pyrazol-4-yl)methyl)thio)-5,5-dimethyl-4,5-dihydroisoxazole and 10 g of sodium tungstate at 20° C. to obtain a reaction mixture. The reaction mixture was heated to 50° C. to obtain a heated reaction mixture. An aqueous solution of 30% hydrogen peroxide (272 g, 2.4 mole) was added dropwise to the heated reaction mixture over 1 hour to obtain a reaction mass. The reaction mass was heated to 50° C. under stirring and continued stirring at 50° C. for 13 hours to obtain a product mass. The pH of the product mass was adjusted to 8 by using 5% NaOH solution at 50° C. After completion of the reaction, an aqueous solution of sodium thiosulphate was added to the product mass to decompose excess of hydrogen peroxide followed by adding 400 ml water slowly to obtain a resultant product mass. The resultant product mass was heated to 70° C. under stirring for 30 minutes followed by cooling to 10° C. and filtered to obtain a cake. The cake was washed with methanol followed by water to obtain a solid product. The solid product was dried under vacuum at 80° C. to obtain pyroxasulfone.

The yield of pyroxasulfone was 90.5% and the purity was 99.1%.

### Experiment 8C: Preparation of Pyroxasulfone from 3-[[5-(difluoromethoxy)-1-methyl-3-(trifluoromethyl)pyrazole-4-yl]methylsulfanyl]-5,5-dimethyl-4H-isoxazole Using Sodium Tungstate as Catalyst and Dichloro Acetic Acid as a Fluid Medium (Step h)

A reactor was charged with 1.9 kg dichloro acetic acid followed by adding 359 g (1 mole) of 3-(((5-(difluoromethoxy)-1-methyl-3-(trifluoromethyl)-1H-pyrazol-4-yl)methyl)thio)-5,5-dimethyl-4,5-dihydroisoxazole and 14.5 g of sodium tungstate at 20° C. to obtain a reaction mixture. The reaction mixture was heated to 50° C. to obtain a heated reaction mixture. An aqueous solution of 50% hydrogen peroxide (251.6, 3.7 mole) was added dropwise to the heated reaction mixture over 1 hour to obtain a reaction mass. The reaction mass was heated to 50° C. under stirring and continued stirring at 50° C. for 5 hours to obtain a product mass. The product mass was heated to 70° C. under stirring for 30 minutes followed by cooling to 10° C. and filtered to obtain a cake. The cake was washed with water followed by hexane to obtain a solid product. The solid product was dried under vacuum at 80° C. to obtain pyroxasulfone.

The yield of pyroxasulfone was 85% and the purity was 99.5%.

### Experiment 8D: Preparation of Pyroxasulfone from 3-[[5-(difluoromethoxy)-1-methyl-3-(trifluoromethyl)pyrazole-4-yl]methylsulfanyl]-5,5-dimethyl-4H-isoxazole Using Sodium Tungstate as Catalyst in a Mixture of Acetic Acid and Dichloro Acetic Acid (in 95:5 Ratio) as the Fluid Medium (Step h)

A reactor was charged with 95:5 ratio of 1.783 kg acetic acid and 0.094 kg dichloro acetic acid followed by adding 359 gm (1.0 mole) of 3-(((5-(difluoromethoxy)-1-methyl-3-(trifluoromethyl)-1H-pyrazol-4-yl)methyl)thio)-5,5-dimethyl-4,5-dihydroisoxazole and 14.5 gm of sodium tungstate at 20° C. to obtain a reaction mixture. The reaction mixture was heated to 50° C. to obtain a heated reaction mixture. An aqueous solution of 50% hydrogen peroxide (170 g, 2.5 mole) was added dropwise to the heated reaction mixture over 1 hour to obtain a reaction mass. The reaction mass was heated to 50° C. under stirring and continued stirring at 50° C. for 8 hours to obtain a product mass. The product mass was cooled to 10° C. and filtered to obtain a cake. The cake was mixed in water followed by adding an aqueous solution of sodium thiosulphate to decompose excess hydrogen peroxide. The cake was washed with water to obtain a solid product. The solid product was dried under vacuum at 80° C. to obtain pyroxasulfone.

The yield of pyroxasulfone was 86% and the purity was 99.5%.

### Experiment 8E: Preparation of Pyroxasulfone from 3-[[5-(difluoromethoxy)-1-methyl-3-(trifluoromethyl)pyrazole-4-yl]methylsulfanyl]-5,5-dimethyl-4H-isoxazole Using Sodium Tungstate as a Catalyst in a Mother Liquor of Chloro Acetic Acid Containing a Mixture of Acetic Acid, Monochloro Acetic Acid, and Dichloro Acetic Acid as the Fluid Medium (Step h)

A reactor was charged with 1.0 kg mother liquor of chloro acetic acid (containing 2% acetic acid, 42.6% monochloro acetic acid, 48.74% dichloro acetic acid, and 6.7% water) followed by adding 359 g (1 mole) of 3-(((5-(difluoromethoxy)-1-methyl-3-(trifluoromethyl)-1H-pyrazol-4-yl)methyl)thio)-5,5-dimethyl-4,5-dihydroisoxazole and 14.5 g of sodium tungstate at 20° C. to obtain a reaction mixture. The reaction mixture was heated to 50° C. to obtain a heated reaction mixture. An aqueous solution of 50% hydrogen peroxide (251.6 g, 3.7 mole) was added dropwise to the heated reaction mixture over 1 hour to obtain a reaction mass. The reaction mass was heated to 50° C. under stirring and continued stirring at 50° C. for 7.5 hours to obtain a product mass. 620 g (34.44 m) of water was added to the product mass to make 57% mother liquor of chloroacetic acid and an aqueous solution of sodium thiosulphate was added to the product mass to decompose excess hydrogen peroxide. The resultant product mass was cooled to 10° C. and filtered to obtain a cake. The cake was washed with water to obtain a solid product. The solid product was dried under vacuum at 80° C. to obtain pyroxasulfone.

The yield of pyroxasulfone was 81% and the purity was 99%.

### Technical Advancement

The present disclosure described hereinabove has several technical advantages including, but not limited to, the realization of a process for the preparation of pyroxasulfone that
proceeds under mild reaction conditions;
is simple, cost-effective, and environment friendly;
provides pyroxasulfone having a comparatively better purity and yield;
wherein the step for the preparation of 5-hydroxy-1-methyl-3-trifluoromethyl-1H-pyrazole is a green process, wherein both the base and the fluid medium used in the reactions can be recovered and recycled;
wherein the step for the preparation of 5-hydroxy-1-methyl-3-trifluoromethyl-1H-pyrazole provides 5-hydroxy-1-methyl-3-trifluoromethyl-1H-pyrazole with comparatively high purity, high yield, and high selectivity; and
wherein the step for the preparation of 5-hydroxy-1-methyl-3-trifluoromethyl-1H-pyrazoleprovides desired isomer content of >99% in the final product.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values given for various physical parameters, dimensions, and quantities are only approximate values and it is envisaged that the values higher than the numerical value assigned to the physical parameters, dimensions, and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A process for the preparation of pyroxasulfone, said process comprising the following steps:
a. reacting ethyl-4,4,4-trifluoroacetoacetate (ETFAA) with a hydrazine salt in a first fluid medium by using a first base at a first predetermined temperature for a first predetermined time period to obtain 5-hydroxy-1-methyl-3-trifluoromethyl-1H-pyrazole;
b. difluoromethylating said 5-hydroxy-1-methyl-3-trifluoromethyl-1H-pyrazole by using a difluoromethylating agent and a second base in a second fluid medium at a second predetermined temperature for a second predetermined time period to obtain 5-(difluoromethoxy)-1-methyl-3-(trifluoromethyl)-1H-pyrazole;
c. chloromethylating said 5-(difluoromethoxy)-1-methyl-3-(trifluoromethyl)-1H-pyrazole by using a chloromethylating agent in the presence of a first catalyst at a third predetermined temperature for a third predetermined time period to obtain 4-(chloromethyl)-5-(difluoromethoxy)-1-methyl-3-(trifluoromethyl)-1H-pyrazole;
d. separately reacting glyoxylic acid with hydroxylamine and a halogenating agent at a fourth predetermined temperature for a fourth predetermined time period to obtain 1,1′-dibromoformaldoxime;
e. reacting said 1,1′-dibromoformaldoxime, a third base, and isobutylene in a third fluid medium at a fifth predetermined temperature for a fifth predetermined time period to obtain 3-bromo-5,5-dimethyl-4,5-dihydroisoxazole;
f. reacting said 3-bromo-5,5-dimethyl-4,5-dihydroisoxazole with thiourea in a fourth fluid medium in the presence of a second catalyst at a sixth predetermined temperature for a sixth predetermined time period to obtain 5,5-dimethyl-4,5-dihydroisoxazole-3-ylisothiourea hydrobromide;
g. reacting said 4-(chloromethyl)-5-(difluoromethoxy)-1-methyl-3-(trifluoromethyl)-1H-pyrazole with said 5,5-dimethyl-4,5-dihydroisoxazole-3-ylisothiourea hydrobromide by using a fourth base in a fifth fluid medium at a seventh predetermined temperature for a seventh predetermined time period to obtain 3-(((5-(difluoromethoxy)-1-methyl-3-(trifluoromethyl)-1H-pyrazol-4-yl)methyl)thio)-5,5-dimethyl-4,5-dihydroisoxazole; and
h. oxidizing said 3-(((5-(difluoromethoxy)-1-methyl-3-(trifluoromethyl)-1H-pyrazol-4-yl)methyl)thio)-5,5-dimethyl-4,5-dihydroisoxazole by using an oxidizing agent in the presence of a third catalyst in a sixth fluid medium at an eighth predetermined temperature for an eighth predetermined time period to obtain pyroxasulfone.

2. The process as claimed in claim 1, wherein said hydrazine salt is at least one selected from the group consisting of methyl hydrazine sulphate, and methyl hydrazine hydrochloride.

3. The process as claimed in claim 1, wherein
i. said first fluid medium is at least one selected from the group consisting of methanol, ethanol, propanol, and n-butanol;
ii. said first base is at least one selected from the group consisting of triethylamine, and diethylamine;
iii. said first predetermined time period is in the range of 4 hours to 8 hours;
iv. said first predetermined temperature is in the range of 30° C. to 90° C.; and
v. a mole ratio of said hydrazine salt to said ethyl-4,4,4-trifluoroacetoacetate (ETFAA) is in the range of 1:1 to 1.5:1.

4. The process as claimed in claim 1, wherein said difluoromethylating agent is chlorodifluoromethane.

5. The process as claimed in claim 1, wherein
i. said second fluid medium is at least one selected from the group consisting of acetonitrile, methanol, ethanol, isopropyl alcohol, tetrahydrofuran (THF), dioxane, monoglyme, and diglyme;
ii. said second base is at least one selected from the group consisting of sodium hydroxide, potassium hydroxide, sodium carbonate, and potassium carbonate;
iii. said second predetermined temperature is in the range of 20° C. to 45° C.;
iv. said second predetermined time period is in the range of 2 hours to 10 hours; and
v. a mole ratio of said difluoromethylating agent to said 5-hydroxy-1-methyl-3-trifluoromethyl-1H-pyrazole is in the range of 1:1 to 2.5:1.

6. The process as claimed in claim 1, wherein said chloromethylating agent is a mixture of a chlorinating agent and an aldehyde.

7. The process as claimed in claim 6, wherein said chlorinating agent is HCl.

8. The process as claimed in claim 6, wherein said aldehyde is at least one selected from the group consisting of paraformaldehyde, and formalin.

9. The process as claimed in claim 1, wherein
i. said first catalyst is at least one selected from the group consisting of methanesulphonic acid, monochloroacetic acid, dichloroacetic acid, trichloroacetic acid, benzenesulfonic acid and p-toluene sulfonic acid;
ii. said third predetermined temperature is in the range of 75° C. to 100° C.;
iii. said third predetermined time period is in the range of 10 hours to 20 hours; and
iv. a mole ratio of said chloromethylating agent to said 5-(difluoromethoxy)-1-methyl-3-(trifluoromethyl)-1H-pyrazole is in the range of 6:1 to 7:1.

10. The process as claimed in claim 1, wherein said halogenating agent is bromine.

11. The process as claimed in claim 1, wherein
i. said fourth predetermined temperature is in the range of −5° C. to 5° C.; and
ii. said fourth predetermined time period is in the range of 4 hours to 6 hours.

12. The process as claimed in claim 1, wherein
i. said fifth predetermined temperature is in the range of 20° C. to 40° C.;
ii. said fifth predetermined time period is in the range of 1 hour to 2 hours;
iii. said third base is at least one selected from the group consisting of sodium bicarbonate, potassium bicarbonate, potassium carbonate, and sodium carbonate; and
iv. said third fluid medium is at least one selected from the group consisting of water, methylene dichloride and ethylene dichloride.

13. The process as claimed in claim 1, wherein said second catalyst is at least one selected from the group consisting of hydrogen bromide, and hydrogen chloride.

14. The process as claimed in claim 1, wherein
i. said fourth fluid medium is at least one selected from the group consisting of methanol, ethanol, isopropyl alcohol, and n-butanol;
ii. said sixth predetermined temperature is in the range of 20° C. to 40° C.; and
iii. said sixth predetermined time period is in the range of 13 hours to 17 hours.

15. The process as claimed in claim 1, wherein
i. said fourth base is at least one selected from the group consisting of sodium hydroxide, potassium carbonate, and pyridine;
ii. said fifth fluid medium is at least one selected from the group consisting of water and methanol;
iii. said seventh predetermined temperature is in the range of 10° C. to 40° C.;
iv. said seventh predetermined time period is in the range of 3 hours to 35 hours; and
v. a mole ratio of said 5,5-dimethyl-4,5-dihydroisoxazole-3-ylisothiourea hydrobromide to said 4-(chloromethyl)-5-(difluoromethoxy)-1-methyl-3-(trifluoromethyl)-1H-pyrazole is in the range of 1:1 to 1.5:1.

16. The process as claimed in claim 1, wherein said sixth fluid medium is at least one selected from the group consisting of dichloro acetic acid, monochloro acetic acid, acetic acid, pivalic acid, formic acid, aliphatic alcohol, and a mother liquor of chloroacetic acid.

17. The process as claimed in claim 1, wherein said third catalyst is at least one selected from the group consisting of phosphotungstic acid, sodium tungstate, methyl tri-n-octyl ammonium hydrogen sulphate, phenyl phosphonic acid, sodium phosphotungstate, phosphomolybdic acid, and silicotungstic acid.

18. The process as claimed in claim 1, wherein said oxidizing agent is hydrogen peroxide.

19. The process as claimed in claim 1, wherein
i. said eighth predetermined temperature is in the range of 40° C. to 80° C.;
ii. said eighth predetermined time period is in the range of 2 hours to 20 hours; and
iii. a mole ratio of said oxidizing agent to said 3-(((5-(difluoromethoxy)-1-methyl-3-(trifluoromethyl)-1H-pyrazol-4-yl)methyl)thio)-5,5-dimethyl-4,5-dihydroisoxazole is in the range of 2:1 to 5:1.
